(19) United States
(12) Patent Application Publication
(10) Pub. No.: US 2013/0312759 A1
(43) Pub. Date: Nov. 28, 2013

(54) AIRTIGHT SUCTION DEVICE WITH AIR SUPPLY FUNCTION AND ROTARY SWITCH THEREOF

(71) Applicant: Hsuan-Chiao HO, Tongluo Township (TW)

(72) Inventor: Hsuan-Chiao HO, Tongluo Township (TW)

(73) Assignee: PACIFIC HOSPITAL SUPPLY CO., LTD.

(21) Appl. No.: 13/480,355

(22) Filed: May 24, 2012

(51) Int. Cl. A61M 1/00; A61M 16/04

(52) U.S. Cl. 128/205.27; 604/540

(57) ABSTRACT

In an airtight suction device and a rotary switch thereof, the airtight suction device includes a main body having a communication space and a plurality of connectors. These connectors include a suction connector, a respiratory connector, a cleaning connector, and a control valve connector. The rotary switch is provided on the control valve connector and includes a rotary cover and a hollow post. The hollow post has a peripheral wall and a blocking piece. The peripheral wall is provided with a pair of suction holes. The blocking piece is provided with a ventilation hole for allowing external air to flow into the suction holes.

### BACKGROUND OF THE INVENTION

1. Field of the Invention

The present invention relates to an airtight suction device, and in particular to an airtight suction device with air supply function and a rotary switch thereof

2. Description of Prior Art

As for a patient who uses an artificial breathing device or a tracheostomy tube, a suction device is used to suck the sputum of the patient while supplying oxygen to the patient. The existing suction device includes a main body, a negative pressure generator and an oxygen supply device both connected to the main body. The main body comprises a trachea connector connected to the trachea of the patient, an oxygen supply connector connected to the oxygen supply device, a suction connector connected to a suction tube, and a cleaning connector connected to a cleaning device.

In practice, when the above-mentioned suction device is used, oxygen is supplied from the oxygen supply device through the oxygen supply connector and the trachea connector into the trachea of the patient, thereby supplying oxygen to the patient necessary for breathing. On the other hand, in order to suck the sputum of the patient, medical personnel has to insert a suction tube into the suction connector and the trachea connector. Then, the negative pressure generator generates a negative pressure to suck the sputum in the mouth or nose of the patient into the suction tube. Thereafter, the medical personnel connects the cleaning device to the cleaning connector, so that the cleaning liquid of the cleaning device can flow into the main body to clean the remaining sputum in the suction device.

However, during the cleaning process of the suction device, a portion of oxygen may be drawn into the cleaning connector due to the negative pressure of the negative pressure generator. As a result, the patient cannot breathe sufficient amount of oxygen, so that the patient may often feel uncomfortable during the cleaning process of the suction device.

In view of the above, the present Inventor proposes a novel and reasonable structure based on his expert knowledge and deliberate researches.

### SUMMARY OF THE INVENTION

The present invention is to provide an airtight suction device with air supply function, which can prevent the change in the flowing direction of oxygen outputted by the oxygen supply device during the cleaning process of the suction tube. Thus, the patient can breathe sufficient amount of oxygen without feeling uncomfortable during the cleaning process of the suction tube.

The present invention provides an airtight suction device with air supply function, which is configured to be inserted by a suction tube. The airtight suction device includes a main body and a rotary switch. The main body includes a communication space and a plurality of connectors communicated with each other via the communication space. These connectors includes a suction connector connected to the suction tube, a trachea connector connected to the trachea of a patient, a respiratory connector connected to an oxygen supply device, a cleaning connector and a control valve connector both oppositely provided between the respiratory connector and the suction connector. The rotary switch is provided on the control valve connector and comprises a rotary cover and a hollow post connected to the rotary cover. The hollow post has a peripheral wall and a blocking piece straddling in the peripheral wall. The peripheral wall of the hollow post is provided with a pair of suction holes at positions corresponding to the communication space for allowing the suction tube to be inserted therein. The blocking piece is provided with a ventilation hole for allowing external air to flow into the pair of suction holes.

The present invention provides a rotary switch of an airtight suction device with air supply function, thereby controlling a cleaning process of a suction tube. The airtight suction device comprises a main body having a control valve connector. The rotary switch is provided on the control valve connector and comprises a rotary cover and a hollow post connected to the rotary cover. The hollow post has a peripheral wall and a blocking piece straddling in the peripheral wall. The peripheral wall is provided with a pair of suction holes for allowing the suction tube to be inserted therein. The blocking piece is provided with a ventilation hole for allowing external air to flow into the pair of suction holes.

In comparison with prior art, the airtight suction device of the present invention is provided with a rotary switch. When the rotary switch is rotated to a shut-off position, the cleaning process of the suction tube can be performed. In order to prevent the change in the flowing direction of oxygen outputted by an oxygen supply device due to the negative pressure generated by the negative pressure generator during the cleaning process of the suction tube, the blocking piece of the rotary switch is provided with a ventilation hole for allowing external air to flow into the rotary switch. In this way, the pressure in the rotary switch can be balanced, so that the patient can still breathe sufficient amount of oxygen during the cleaning process of the suction tube. Therefore, the present invention has an improved practicability.

### BRIEF DESCRIPTION OF DRAWING

FIG. 1 is an exploded perspective view showing the main body and the rotary switch of the present invention;

FIG. 2 is a perspective view showing the external appearance after the main body and the rotary switch of the present invention are assembled together;

FIG. 3 is a schematic view showing the operation of the airtight suction device of the present invention;

FIG. 4 is a schematic view showing the operation of the rotary switch of the present invention;

FIG. 5 is another schematic view showing the operation of the rotary switch of the present invention;

FIG. 6 is a schematic view showing one aspect of the ventilation hole provided in the rotary switch of the present invention; and

FIG. 7 is a schematic view showing another aspect of the ventilation hole provided in the rotary switch of the present invention.

### DETAILED DESCRIPTION OF THE INVENTION

The detailed description and technical contents of the present invention will become apparent with the following detailed description accompanied with related drawings. It is noteworthy to point out that the drawings is provided for the illustration purpose only, but not intended for limiting the scope of the present invention.

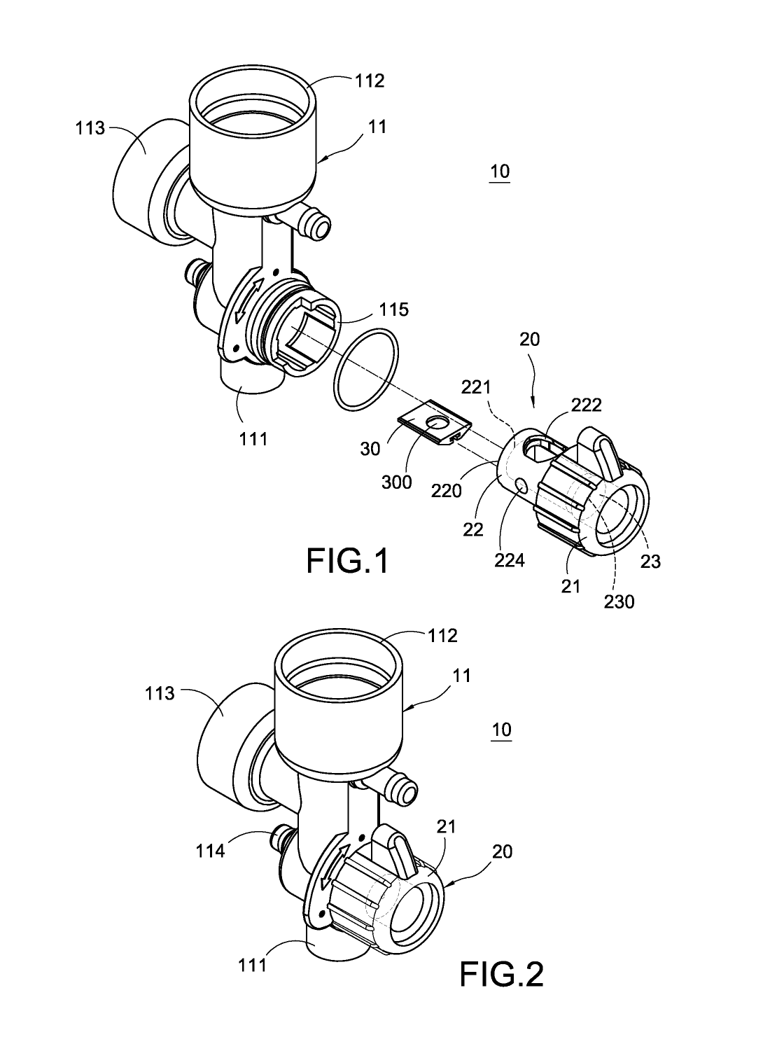

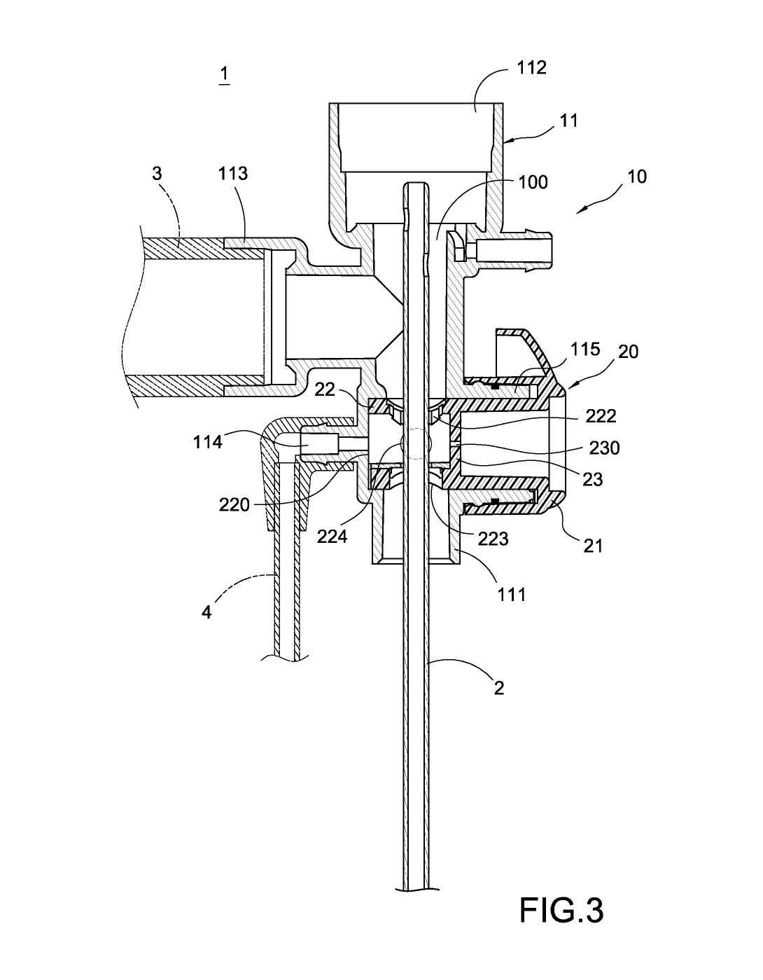

Please refer to FIGS. 1 to 3. FIG. 1 is an exploded perspective view showing the main body and the rotary switch of the present invention. FIG. 2 is a perspective view showing the external appearance after the main body and the rotary switch of the present invention are assembled together. FIG. 3 is a schematic view showing the operation of the airtight suction device of the present invention. The present invention provides an airtight suction device 1 with air supply function, which is inserted by a suction tube 2. The airtight suction tube 1 includes a main body 10, a rotary switch 20, and a sputum-wiping piece 30.

The main body 10 includes a communication space 100 and a plurality of connectors 11 communicated with each other via the communication space 100. These connectors 11 comprises a suction connector 111 connected to the suction tube 2, a trachea connector 112 connected to the trachea of the patient, a respiratory connector 113 connected to an oxygen supply device 3, a cleaning connector 114 and a control valve connector 115 both oppositely provided between the respiratory connector 113 and the suction connector 111. The cleaning connector 114 is connected to a cleaning device 4.

Further, the rotary switch 20 is provided on the control valve connector 115. The rotary switch 20 comprises a rotary cover 21 and a hollow post 22 connected to the rotary cover 21. The hollow post 22 has a peripheral wall 221 and a blocking piece 23 straddling in the peripheral wall 221.

When the rotary switch 20 is assembled with the control valve connector 115, the rotary cover 21 is put on the outer periphery of the control valve connector 115. The hollow post 22 is inserted into the communication space 110 along the control valve connector 115 and thus positioned at one side of the cleaning connector 114. At this time, a port 220 provided at one end of the hollow post 22 is in communication with the cleaning connector 114. Further, the peripheral wall 221 is provided with a pair of sucking holes 222, 223 at positions corresponding to the communication space 100 for allowing the suction tube 2 to be inserted therein. Further, the hollow post 22 has a cleaning liquid exit 224 provided on the peripheral wall 221 and located between the pair of suction holes 222 and 223.

It should be noted that, the blocking piece 23 of the rotary switch 20 is provided with a ventilation hole 230 for allowing external air to flow into the pair of suction holes 222 and 223, thereby achieving the air supply function.

The sputum-wiping piece 30 is provided between the pair of suction holes 222 and 223. The sputum-wiping piece 30 is provided with a via hole 300 at a position corresponding to the positions of the pair of suction holes 222 and 223. In practice, the sputum-wiping piece 30 is fixedly inserted into the peripheral wall 221 of the hollow post 22 with its one side abutting against the blocking piece 23.

In order to suck the sputum of the patient, the suction tube 2 is inserted into the lower suction hole 222 near the suction connector 111, and then passes through the via hole 300 and the upper suction hole 223 near the trachea connector 112. Finally, the suction tube 2 is inserted into the trachea connector 112. At this time, the suction tube 2 can suck the sputum contained in the mouth or nose of the patient by means of the negative pressure generated by the negative pressure generator.

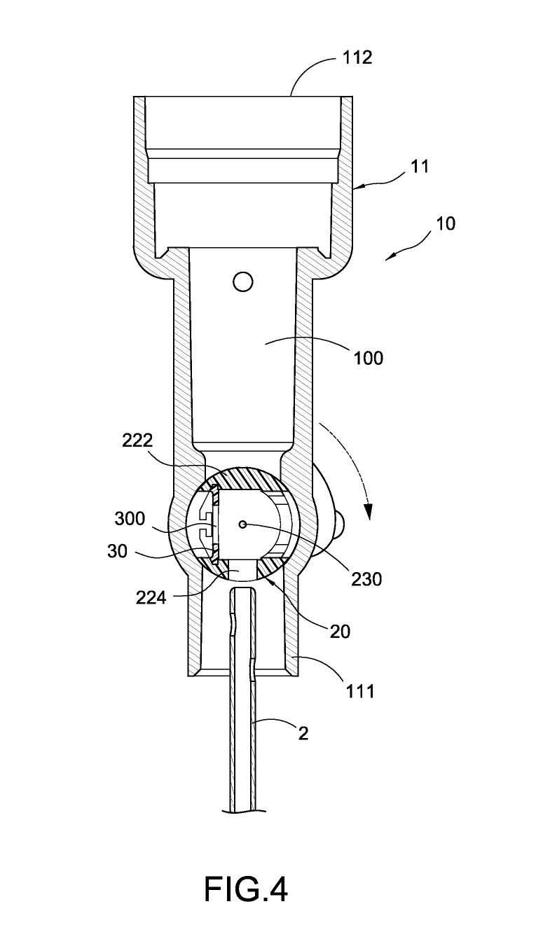

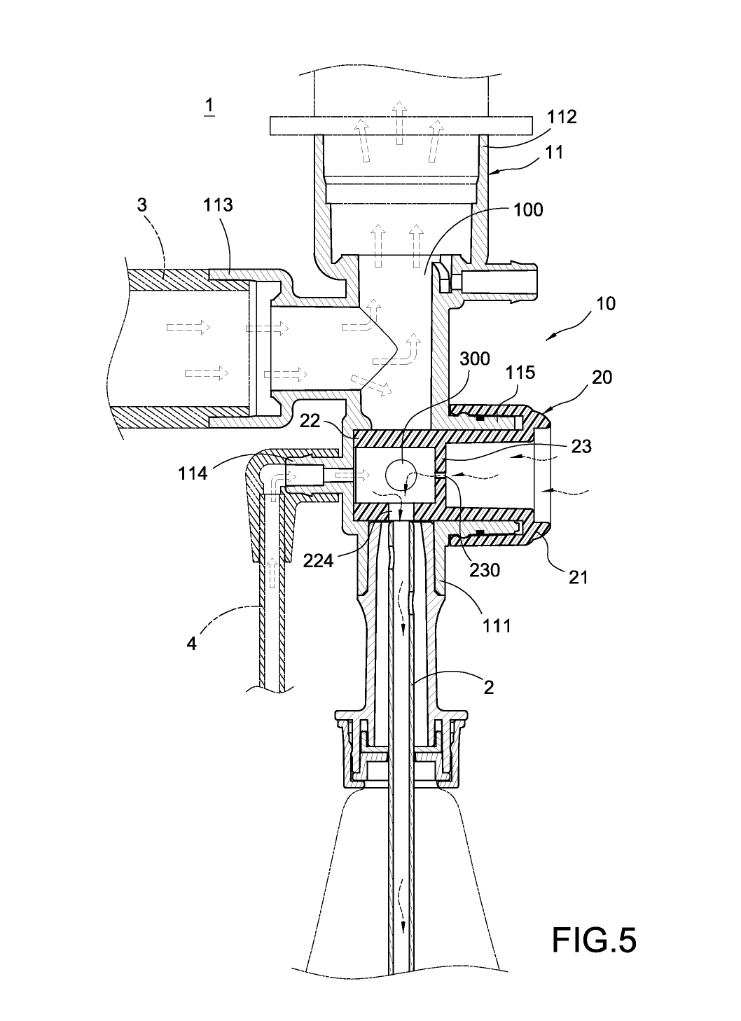

Please refer to FIGS. 4 and 5. FIG. 4 is a schematic view showing the operation of the rotary switch of the present invention, and FIG. 5 is another schematic view showing the operation of the rotary switch of the present invention. After completing the suction process, the medical personnel connects the cleaning device 4 to the cleaning connector 114, thereby cleaning the remaining sputum in the airtight suction device 1.

In order to perform the cleaning process of the suction tube 2, the suction tube 2 has to be drawn out of the trachea connector 112 and the pair of suction holes 222 and 223. When the suction tube 2 is drawn out of the suction holes 222 and 223, the sputum adhered on the outer surface of the suction tube 2 will be wiped away by the sputum-wiping piece 30 to stay in the rotary switch 20.

Then, the medical personnel rotates the rotary switch 20 by approximate 90 degrees to make the cleaning liquid exit 224 of the hollow post 22 to face the suction connector 111. At this time, the medical personnel turns on the cleaning device 4 to thereby allow the cleaning liquid to flow from the cleaning device 4 into the cleaning connector 114. Finally, the used cleaning liquid is exhausted from the cleaning liquid exit 224 and the suction tube 2. At this time, the negative pressure generator of the suction tube 2 continuously generates a negative pressure to thereby suck the remaining sputum and the used cleaning liquid stayed in the hollow post 22 and the suction tube 2.

It should be noted that, during the cleaning process of the suction tube 2, the external air will enter the hollow post 22 via the ventilation hole 230 of he blocking piece 23, thereby balancing the pressure in the hollow post 22. In this way, the change in the flowing direction of oxygen outputted from the oxygen supply device 3 can be prevented, so that the patient can still breathe sufficient amount of oxygen.

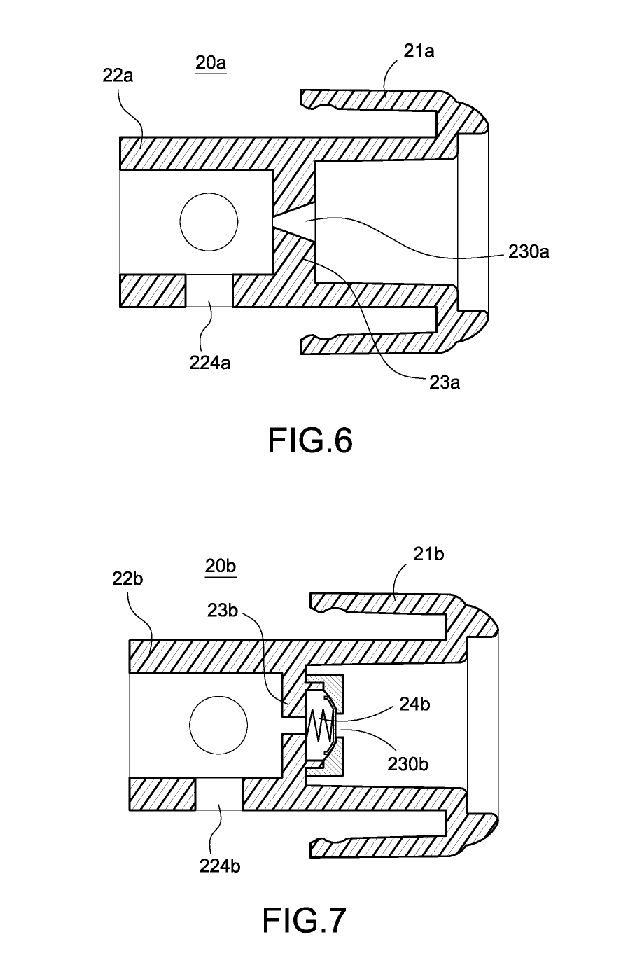

Please refer to FIGS. 6 and 7, which are schematic views showing two aspects of the ventilation hole provided in the rotary switch of the present invention respectively. In FIG. 6, the rotary switch 20a comprises a rotary cover 21 a and a hollow post 22a connected to the rotary cover 21a. The hollow post 22a has a blocking piece 23a and a cleaning liquid exit 224a. The blocking piece 23a is provided with a ventilation hole 230a configured as a conical hole. The diameter of the ventilation 230a gradually increases toward the rotary cover 21a, thereby facilitating external air to flow in and preventing the internal liquid from flowing out.

Similarly, in FIG. 7, the rotary switch 20b comprises a rotary cover 21b and a hollow post 22b connected to the rotary cover 21b. The hollow post 22b has a blocking piece 23b and a cleaning liquid exit 224b. The blocking piece 23b is provided with a ventilation hole 230b. The difference between FIG. 7 and FIG. 6 lies in that the rotary switch 20b further includes a check valve 24b provided on the ventilation hole 230b, thereby allowing external air to flow in and preventing the internal liquid from flowing out.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. An airtight suction device with air supply function, configured to be inserted by a suction tube, the airtight suction device including:
a main body comprising a communication space and a plurality of connectors communicated with each other via the communication space, these connectors comprising: a suction connector connected to the suction tube, a trachea connector connected to the trachea of a patient, a respiratory connector connected to an oxygen supply device, a cleaning connector and a control valve connector both oppositely provided between the respiratory connector and the suction connector; and
a rotary switch provided on the control valve connector and comprising a rotary cover and a hollow post connected to the rotary cover, the hollow post having a peripheral wall and a blocking piece straddling in the peripheral wall, the peripheral wall of the hollow post being provided with a pair of suction holes at positions corresponding to the communication space for allowing the suction tube to be inserted therein,
wherein the blocking piece is provided with a ventilation hole for allowing external air to flow into the pair of suction holes.

2. The airtight suction device with air supply function according to claim 1, further including a sputum-wiping piece provided between the pair of suction holes in the hollow post, the blocking piece being provided with a via hole at a position corresponding to the pair of suction holes.

3. The airtight suction device with air supply function according to claim 2, wherein one side of the sputum-wiping piece abuts against the blocking piece.

4. The airtight suction device with air supply function according to claim 1, wherein the ventilation hole is a conical hole.

5. The airtight suction device with air supply function according to claim 4, wherein the diameter of the ventilation hole gradually increases toward the rotary cover.

6. The airtight suction device with air supply function according to claim 1, wherein the hollow post has a cleaning liquid exit provided on the peripheral wall and located between the pair of suction holes.

7. The airtight suction device with air supply function according to claim 1, wherein the rotary cover is put on the outer periphery of the control valve connector, the hollow post is inserted into the communication space along the control valve connector and located on one side of the cleaning connector.

8. The airtight suction device with air supply function according to claim 7, wherein a port is provided at one end of the hollow post in communication with the cleaning connector.

9. The airtight suction device with air supply function according to claim 1, wherein the rotary switch further includes a check valve provided on the ventilation hole.

10. A rotary switch of an airtight suction device with air supply function, configured to control a cleaning process of a suction tube, the airtight suction device comprising a main body having a control valve connector, the rotary switch being provided on the control valve connector and comprising a rotary cover and a hollow post connected to the rotary cover, the hollow post having a peripheral wall and a blocking piece straddling in the peripheral wall, the peripheral wall being provided with a pair of suction holes for allowing the suction tube to be inserted therein, the blocking piece being provided with a ventilation hole for allowing external air to flow into the pair of suction holes.

11. The rotary switch of an airtight suction device with air supply function according to claim 10, wherein the ventilation hole is a conical hole.

12. The rotary switch of an airtight suction device with air supply function according to claim 11, wherein the diameter of the ventilation hole gradually increases toward the rotary cover.

13. The rotary switch of an airtight suction device with air supply function according to claim 10, wherein the hollow post has a cleaning liquid exit provided on the peripheral wall and located between the pair of suction holes.

14. The rotary switch of an airtight suction device with air supply function according to claim 10, wherein the rotary cover is put on the outer periphery of the control valve connector, the hollow post is inserted into the communication space along the control valve connector and located on one side of the cleaning connector.

15. The rotary switch of an airtight suction device with air supply function according to claim 14, wherein a port is provided at one end of the hollow post in communication with the cleaning connector.

16. The rotary switch of an airtight suction device with air supply function according to claim 10, wherein the rotary switch further includes a check valve provided on the ventilation hole.
